(19) United States
(12) Patent Application Publication
(10) Pub. No.: US 2026/0199285 A1
(43) Pub. Date: Jul. 16, 2026

(54) TAXANE FORMULATIONS

(71) Applicant: BIOPURIFICATION, LLC, Laguna Niguel, CA (US)

(72) Inventor: Bahman Shimiaei, Laguna Niguel, CA (US)

(21) Appl. No.: 19/134,566

(22) PCT Filed: Dec. 22, 2023
(86) PCT No.: PCT/US2023/085596

§ 371 Date: May 30, 2025

Related U.S. Application Data
(60) Provisional application No. 63/434,903, filed on Dec. 22, 2022.

(51) Int. Cl. A61K 31/337; A61K 9/16; A61K 9/19; A61K 47/40

(52) U.S. Cl. CPC A61K 31/337; A61K 9/1623; A61K 9/1635; A61K 9/19; A61K 47/40

(57) ABSTRACT

Provided is a pharmaceutically acceptable formulation comprising a taxane and an acid-activated 2-hydroxypropyl-β-cyclodextrin (CD). Also provided is a method of treating a patient with a taxane. The method comprises infusing the above formulation into the patient. Additionally provided is a method of preparing the above formulation. Further provided is an infusion bag comprising the above formulation.

### CROSS-REFERENCE TO RELATED APPLICATIONS

This application claims the benefit of U.S. Provisional Application No. 63/434,903, filed Dec. 22, 2022, and incorporated by reference herein in its entirety.

### BACKGROUND OF THE INVENTION

### (1) Field of the Invention

The present application generally relates to pharmaceutical formulations and methods of preparing those formulations. More specifically, taxane formulations utilizing hydroxypropyl-β-cyclodextrins, and methods of preparing the taxane formulations are provided.

### (2) Description of the Related Art

Antineoplastic agents inhibit and combat the development of neoplasms, which are abnormal masses of tissue resulting from irregular proliferation of cells. One class of antineoplastic agents are taxanes, originally derived from the renewable needle biomass of yew plants.

Taxanes that are approved for cancer treatment include docetaxel, paclitaxel and cabazitaxel.

Docetaxel binds to free tubulin and promotes the assembly of microtubules, which reduces the availability of tubulin for, and thereby prevents, cell division. Simultaneously, docetaxel inhibits microtubule disassembly, causing apoptosis. See TAXOTERE® Prescribing Information.

Docetaxel is marketed as TAXOTERE®, which is FDA approved for breast cancer, non-small cell lung cancer, hormone refractory prostate cancer, gastric adenocarcinoma, and squamous cell carcinoma of head and neck cancer. TAXOTERE® is available as an injection concentrate in single-dose vials containing 40 mg/mL docetaxel (on an anhydrous basis) and 1040 mg/mL polysorbate 80 (also known as TWEEN-80®). The polysorbate 80 is utilized to solubilize the highly hydrophobic taxane compound. TAXOTERE® is provided as an injection concentrate and requires dilution to 10 mg/mL prior to use. Hence, a sterile, non-pyrogenic, single-dose diluent containing 13% ethanol in water for injection is supplied in a separate vial.

The anti-tumor mechanism of docetaxel is the same as that of other taxanes, which exert their anti-tumor activity by inhibiting tubulin depolymerization. However, docetaxel's inhibitory activity of tubulin depolymerization is about 2 times of that of paclitaxel, so it has a better curative effect on advanced breast carcinoma, superior to other single drug therapies (25%-53%) and stronger than anthracyclines, with an effective ratio of 49% for treatment of advanced breast carcinoma and better curative effects on advanced non-a-cell lung cancer, advanced ovarian cancer, pancreatic cancer, head and neck cancer and gastric cancer.

After dilution, the required amount of docetaxel is transferred from the 10 mg/mL initial diluted solution to an infusion bag or bottle of either 0.9% sodium chloride solution or 5% dextrose solution to produce a final dilution for infusion having a concentration of 0.3 to 0.74 mg/mL. The recommended therapy is six cycles of docetaxel given once every three weeks.

The presence of polysorbate 80 in TAXOTERE®, however, most often results in serious side effects. Such reactions characterized by generalized rash/erythema, hypotension and/or bronchospasm, or very rarely fatal anaphylaxis, have been reported in patients. Hypersensitivity reactions require immediate discontinuation of the TAXOTERE® infusion and administration of appropriate therapy. See Schwartzberg and Navari, 2018.

In order to reduce the side effects induced by polysorbate 80, patients are treated with dexamethasone for three days prior to therapy. Dexamethasone is a steroid that suppresses the immune response in patients, which can be especially detrimental in cancer patients under chemotherapy, whose immunity may already be compromised due to the destruction of healthy cells by the chemotherapeutic treatment. As a result, these patients can be susceptible to bacterial and fungal infections. Further, despite receiving the recommended 3-day dexamethasone premedication, patients still report hypersensitivity side effects from TAXOTERE®. Due to these side effects, most of the patients stop TAXOTERE® therapy by the end of the second or third cycle, skip a dose, or continue further therapy at a reduced dose. The currently marketed formulation of cabazitaxel, JEVTANA®, also has polysorbate 80. Further, other solubilizing agents such as CREMOPHOR EL®, which is a polyethoxylated castor oil used in connection with the marketed paclitaxel product TAXOL®, induce similar allergic reactions requiring premedication with a steroid. Therefore, new formulations of taxanes are needed to avoid these side effects, premedication requirements, and patient noncompliance issues associated with the currently marketed formulation of TAXOTERE®.

There are other taxane formulations that do not require a surfactant such as polysorbate 80 or polyethoxylated castor oil. See, e.g., WO 99/24073 and references cited in the International Search Report from that application; U.S. Pat. No. 8,481,511; Subramanian et al., 2020. The present invention provides additional new formulations that are simpler to prepare than the above and do not require the use of highly substituted cyclodextrin cellulosic polymers.

### BRIEF SUMMARY OF THE INVENTION

The present invention is based in part on the discovery of taxane formulations that do not require a surfactant such as polysorbate 80 or polyoxyethylated castor oil to achieve a stable formulation that is suitable for infusion.

It is therefore an object of the invention to provide taxane formulations suitable for injection with no surfactant.

It is a further object of the invention to provide taxane formulations suitable for injection with little or no alcohol.

Still another object of the invention is to provide a taxane liquid concentrates that have no surfactant.

It is yet another object of the invention to provide a taxane formulations that have fewer hypersensitivity reactions than the currently commercially available formulations, which currently available formulations have a surfactant component.

An even further object of the invention is to provide taxane lyophilizates for reconstitution where the lyophilizate is totally free of surfactant.

Yet another object of the invention is to provide formulations, liquid concentrates, lyophilizates, etc. containing taxanes that are substantially free or totally free of any highly substituted cyclodextrin cellulosic polymer.

Another object of the invention is to provide a means to administer taxanes to patients without the need for administering dexamethasone or any other steroid and/or without the need to administer an antihistamine prior to the initiation of the docetaxel administration.

An even further object of the invention is to provide a means to administer taxanes to patients without the need for administering dexamethasone or any other steroid and/or without the need to administer an antihistamine prior to the initiation of the taxane administration and without the need for administering dexamethasone or any other steroid or antihistamine during or after the taxane administration.

Still further objects of the invention will be appreciated by those of ordinary skill in the art.

Thus, in some embodiments, a pharmaceutically acceptable formulation comprising a taxane and an acid-activated 2-hydroxypropyl-β-cyclodextrin (CD) is provided.

Also provided is a method of treating a patient with a taxane. The method comprises infusing the above formulation into the patient.

Additionally provided is a method of preparing the above formulation. The method comprises
(a) dissolving the taxane in a solvent;
(b) adding an acid to a solution of 2-hydroxypropyl-β-cyclodextrin (CD) to create acid-activated CD; and
(c) combining the dissolved taxane with the acid-activated CD to create the pharmaceutically acceptable formulation.

Further provided is an infusion bag comprising the above formulation.

### DETAILED DESCRIPTION OF THE INVENTION

Applicant has developed taxane formulations that do not require a surfactant such as polysorbate 80 or polyoxyethylated castor oil to achieve a stable formulation that is suitable for infusion.

Thus, in some embodiments, a pharmaceutically acceptable formulation comprising a taxane and an acid-activated 2-hydroxypropyl-β-cyclodextrin (CD) is provided.

The taxane in these formulations can be any taxane now known or later discovered. As used herein, a taxane is a diterpene having the general chemical structure I, and activity of disrupting microtubule function in mammalian cells.

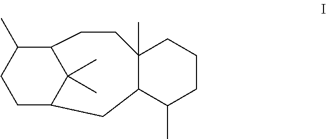 [SMILES: CC1CCCC2(C)CCC3C(C)CCC(CC12)C3(C)C]

Nonlimiting examples of taxanes are paclitaxel, docetaxel, cabazitaxel, larotaxel, ortataxel, BMS-184476, tesetaxel, milataxel, taxoprexin, opaxio, ANG-1005, SB-T-1214, SB-T-1216, SB-T-121602, SB-T-12854, DHA-SB-T-1214, abeo-taxane 15a.2, docetaxel-d₉-t-Boc, cabazitaxel-7,10-d₆, docetaxel-f3-t-Boc, and conjugates thereof. See, e.g., Ojima et al., 2016. In some embodiments, the taxane is docetaxel, paclitaxel or cabazitaxel. In specific embodiments, the taxane is docetaxel.

Surprisingly, the presence of acid-activated CD solubilizes the taxane enough such that a surfactant such as polysorbate 80 or polyoxyethylated castor oil is not necessary to provide a soluble, stable taxane formulation. As used herein, acid-activated CD is not a surfactant.

Thus, in some embodiments, the formulations provided herein do not have a surfactant. Additionally, the formulations provided herein do not require a highly substituted cyclodextrin cellulosic polymer to be stable and soluble. Thus, in various embodiments, the does not have a substituted cyclodextrin cellulosic polymer.

The CD can be activated by any acid. Nonlimiting examples of suitable acids are malonic acid, citric acid, tartartic acid, glutamic acid, phthalic acid, azelaic acid, barbituric acid, benzilic acid, cinnamic acid, fumaric acid, glutaric acid, gluconic acid, hexanoic acid, lactic acid, malic acid, oleic acid, folic acid, propiolic acid, propionic acid, rosolic acid, stearic acid, tannic acid, trifluoroacetic acid. uric acid, ascorbic acid, gallic acid, acetylsalicylic acid, acetic acid, sulfurous acid, sulfuric acid, hyposulfurous acid, persulfuric acid, pyrosulfuric acid, disulfurous acid, dithionous acid, tetrathionic acid, thiosulfurous acid, hydrosulfuric acid, peroxydisulfuric acid, perchloric acid, hydrochloric acid, hypochlorous acid, chlorous acid, chloric acid, hyponitrous acid, nitrous acid, nitric acid, pernitric acid, carbonous acid, carbonic acid, hypocarbonous acid, percarbonic acid, oxalic acid, acetic acid, phosphoric acid, phosphorous acid, hypophosphous acid, perphosphoric acid, hypophosphoric acid, pyrophosphoric acid, hydrophosphoric acid, hydrobromic acid, bromous acid, bromic acid, hypobromous acid, hypoiodous acid, iodous acid, iodic acid, periodic acid, hydroiodic acid, fluorous acid, fluoric acid, hypofluorous acid, perfluoric acid, hydrofluoric acid, chromic acid, chromous acid, hypochromous acid, perchromic acid, hydroselenic acid, selenic acid, selenous acid, hydronitric acid, boric acid, molybdic acid, perxenic acid, silicofluoric acid, telluric acid, tellurous acid, tungstic acid, xenic acid, formic acid, pyroantimonic acid, permanganic acid, manganic acid, antimonic acid, antimonous acid, silicic acid, titanic acid, arsenic acid, pertechnetic acid, hydroarsenic acid, dichromic acid, tetraboric acid, metastannic acid, hypooxalous acid, ferricyanic acid, cyanic acid, silicous acid, hydrocyanic acid, thiocyanic acid, uranic acid, and diuranic acid, as those compounds are commonly known. See, e.g., www.endmemo.com/chem/acidslist.php. The suitability of any particular acid for activating CD for any particular formulation of any particular taxane can be determined without undue experimentation. In some embodiments, the CD is acid-activated with citric acid, acetic acid, hydrochloric acid, ascorbic acid, or glutamic acid. In specific embodiments, the CD is acid-activated with citric acid.

In various embodiments, the formulation is dried, e.g., by lyophilization (for example as described in Example 2), spray drying or solid dispersion drying.

The dried (e.g., lyophilized) formulation is very stable. In some embodiments, the lyophilized formulation is stable for at least 24 months at 2-8° C. In other embodiments, the lyophilized formulation is stable for at least 6 months at room temperature.

In some embodiments, the dried formulation is reconstituted in a liquid suitable for intravenous infusion. Nonlimiting examples of such liquids are normal saline, 5% dextrose, or water for injection.

The above-described formulations, either before drying (e.g., lyophilizing) or reconstituted from a dried state, can be provided sterile and in any concentration. In some embodiments, the formulation is at a concentration of at least 10 mg taxane/ml. In other embodiments, the formulation is at a concentration of less than 10 mg taxane/ml, for example at a concentration of less than 1 mg/ml for infusion. In some of these embodiments, the taxane is docetaxel or cabazitaxel, and the formulation is without a polysorbate (e.g., polysorbate 80) or another surfactant, e.g., polyoxyethylated castor oil. In other embodiments, the liquid formulation is without alcohol.

In various embodiments, these liquid formulations are stable for at least 2 hours, 6 hours, 12 hours, 18 hours, 24 hours, 30 hours, 36 hours, 42 hours, or 48 hours.

Also provided herewith is a method of treating a patient with a taxane. The method comprises infusing the above liquid formulation into the patient. In various embodiments, the formulation has a taxane concentration of less than 1 mg/ml during infusion.

Any taxane now known or later discovered can be utilized in these methods. In some of these embodiments, the taxane is docetaxel, paclitaxel or cabazitaxel. In specific embodiments, the taxane is docetaxel.

In some of these embodiments, the formulation does not comprise a surfactant, for example either a polysorbate such as polysorbate 80, or polyoxyethylated castor oil. In some of those embodiments, the patient was not administered a steroid in preparation for the infusion. In other embodiments, the patient was administered a dosage of a steroid that is reduced, when compared to the required dosage when a formulation comprising a surfactant is infused.

In other embodiments, the patient is administered a formulation without a surfactant and the patient is not administered an antihistamine in preparation for the infusion.

The present invention is also directed to a method of preparing any of the above formulations. The method comprises
(a) dissolving the taxane in a solvent;
(b) adding an acid to a solution of 2-hydroxypropyl-β-cyclodextrin (CD) to create acid-activated CD; and
(c) combining the dissolved taxane with the acid-activated CD to create the pharmaceutically acceptable formulation.

In some of these embodiments, the solvent used to dissolve the taxane in (a) is an alcohol, for example ethanol.

In various embodiments, in (b), the CD is dissolved in water. Further in step (b), any acid can be used to create the acid-activated CD, as previously discussed. In some of these embodiments, the acid is citric acid, acetic acid, hydrochloric acid, ascorbic acid, or glutamic acid. In specific embodiments, the acid is citric acid.

The CD and acid can be at any concentration when combined. In some embodiments, the concentration of CD before adding the acid is at least 50, 100, 200 or 400 mg/ml. In some of these embodiments, the concentration of the acid is at least 0.25, 0.5, 1 or 2 mg/ml or any concentration in between.

In various embodiments, a sugar is added to the acid-activated CD before (c). Any sugar can be added here. In some embodiments, the sugar is dextrose. The sugar may be added at any concentration. In some embodiments, the sugar is added to a concentration of at least 5, 10, 20, 30, 40 or 50 mg/ml or any concentration in between.

In additional embodiments, polyvinyl pyrrolidone (PVP) is added to the acid-activated CD before (c). The PVP may be added at any concentration. In some embodiments, the PVP is add to a concentration of at least 1, 2, 3, 5, 7 or 10 mg/ml, or any concentration in between. In other embodiments, PVP is not added to the acid-activated CD.

In various embodiments, the formulation is dried after (c) by removing the solvent and water. The solvent and water can be removed by any means, for example by lyophilization, spray drying, or solid dispersion drying. In specific embodiments, the solvent and water are removed by lyophilization, e.g., as described in Example 2 below. In some of these embodiments, the dried formulation is reconstituted, e.g., into any liquid suitable for infusion, such as normal saline, 5% dextrose or water for injection.

Also provided is an infusion bag comprising any of the sterile liquid formulations described above.

Preferred embodiments are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### Example 1. Process for Manufacturing a Docetaxel Formulation

Twenty (20) mg of docetaxel trihydrate (DTX) was combined with 0.2 mL ethanol with mixing at ambient temperature until the DTX was completely dissolved.

Twelve hundred (1,200) mg of 2-hydroxypropyl-β-cyclodextrin was combined with 3 mL of purified water at ambient temperature and mixed until completely dissolved (CD solution).

Six (6) mg of citric acid was added to the CD solution and mixed until completely dissolved (CD/citric acid solution).

One hundred fifty 150 mg of dextrose was added to the CD/citric acid solution and mixed at ambient temperature until completely dissolved (CD/citric acid/dextrose solution).

The DTX solution was added to the CD/citric acid/dextrose solution and mixed until completely dissolved. The mixture was clear. The resulting solution (bulk solution) was a stable complexation of docetaxel and acid-activated 2-hydroxypropyl-β-cyclodextrin. The DTX in the solution was stable for 30 days at room temperature.

The above bulk solution was filtered through a 0.2 μm filter then lyophilized to a concentrated powder. The DTX in the lyophilized powder was stable for the indicated shelf-life of the original DTX product.

### Example 2. Lyophilization Cycle

Table 1 reflects the lyophilization procedure used to freeze-dry the bulk solution described in Example 1.

TABLE 1
Lyophilization steps

| Step | Temperature | | Pressure | Duration | |
|---|---|---|---|---|---|
| Freezing | −50° | C. | Atmospheric | One | hour |
| Freeze Hold | −50° | C. | Atmospheric | Four | hours |
| Primary Drying Ramp | −45° | C. | 60 mTorr | 30 | minutes |
| Primary Hold | −45° | C. | 60 mTorr | 120 | hours |
| Secondary Drying Ramp | +25° | C. | 60 mTorr | Two | hours |
| Secondary Drying Hold | +25° | C. | 60 mTorr | 12 | hours |
| Stoppering with | +25° | C. | Atmospheric | | |
| Nitrogen backfill | | | | | |

The lyophilized product was stable for 24 months at 2-8° C.

### REFERENCES

Jevtana® label.
Ojima, I. et al., 2016, Expert Opin Ther Pat. 26: 1-20.
Taxol® label.
Taxotere® label.
Schwartzberg, L S and Navari, R M, 2018, Adv. Ther. 35:754-767.
Subramanian, S. et al., 2020, Breast Cancer: Targets and Therapy 12:77-85.
PCT Patent Publication WO 99/24073.
U.S. Pat. No. 8,481,511 In view of the above, it will be seen that several objectives of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification, including but not limited to patent publications and non-patent literature, and references cited therein, are hereby incorporated by reference. The discussion of the references herein is intended merely to summarize the assertions made by the authors and no admission is made that any reference constitutes prior art. Applicants reserve the right to challenge the accuracy and pertinence of the cited references.

As used herein, in particular embodiments, the terms “about” or “approximately” when preceding a numerical value indicates the value plus or minus a range of 10%. Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The indefinite articles “a” and “an,” as used herein in the specification and in the embodiments, unless clearly indicated to the contrary, should be understood to mean “at least one.”

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements can optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

What is claimed is:

1. A pharmaceutically acceptable formulation consisting essentially of a taxane and an acid-activated 2-hydroxypropyl-β-cyclodextrin (CD).

2. The formulation of claim 1, wherein the taxane is docetaxel, paclitaxel or cabazitaxel.

3. The formulation of claim 1, wherein the taxane is docetaxel.

4-6. (canceled)

7. The formulation of claim 1, wherein the CD is acid-activated with citric acid, acetic acid, hydrochloric acid, ascorbic acid, or glutamic acid.

8. The formulation of claim 1, wherein the CD is acid-activated with citric acid.

9. The formulation of claim 1, lyophilized.

10. The formulation of claim 9, stable for at least 24 months at 2-8° C.

11. The formulation of claim 9, stable for at least 6 months at room temperature.

12. The formulation of claim 9, reconstituted.

13. The formulation of claim 12, reconstituted in normal saline, 5% dextrose, or water for injection.

14-21. (canceled)

22. A method of treating a patient with a taxane, the method comprising infusing the formulation of claim 12 into the patient.

23-48. (canceled)

49. A pharmaceutically acceptable formulation comprising a taxane, an acid-activated 2-hydroxypropyl-β-cyclodextrin (CD) and a sugar.

50. The formulation of claim 49, consisting essentially of a taxane, an acid-activated 2-hydroxypropyl-β-cyclodextrin (CD) and a sugar.

51. The formulation of claim 49, consisting of a taxane, an acid-activated 2-hydroxypropyl-β-cyclodextrin (CD) and a sugar.

52. The formulation of claim 49, without a surfactant, polysorbate 80, polyoxyethylated castor oil, a substituted cyclodextrin cellulosic polymer and alcohol.

53. The formulation of claim 49, wherein the sugar is dextrose.

54. A pharmaceutically acceptable formulation comprising a taxane and an acid-activated 2-hydroxypropyl-β-cyclodextrin (CD) without a surfactant, polysorbate 80, polyoxyethylated castor oil, a substituted cyclodextrin cellulosic polymer and alcohol.

55. The formulation of claim 54, further comprising a sugar.
